(19) United States
(12) Patent Application Publication
(10) Pub. No.: US 2009/0297581 A1
(43) Pub. Date: Dec. 3, 2009

(54) MEDICAL DEVICES HAVING ELECTRODEPOSITED COATINGS

(75) Inventors: Liliana Atanasoska, Edina, MN (US); Wayne Falk, Alameda, CA (US); Michele Zoromski, Minneapolis, MN (US); Robert W. Warner, Woodbury, MN (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/472,234

(22) Filed: May 26, 2009

Related U.S. Application Data
(60) Provisional application No. 61/056,561, filed on May 28, 2008.

(51) Int. Cl. A61F 2/06

(52) U.S. Cl. 424/423; 623/1.46; 977/906

(57) ABSTRACT

According to one aspect, the present invention provides implantable or insertable medical devices that comprise a conductive substrate and an electrodeposited coating over the substrate. The electrodeposited coating includes (a) one or more types of inorganic materials, (b) one or more types of polymeric materials and (c) optionally, one or more types of therapeutic agents. Still other aspects of the invention concern methods of making and using such devices.

### RELATED APPLICATIONS

This application claims priority from U.S. provisional application 61/056,561, filed May 28, 2008, which is incorporated by reference herein in its entirety.

### FIELD OF THE INVENTION

The present invention relates to medical devices and more particularly to implantable or insertable medical devices having electrodeposited coatings.

### BACKGROUND OF THE INVENTION

Implantable and insertable medical devices are commonly provided with one or more coatings which may serve a wide variety of functions including, for example, providing lubricity, imparting biocompatibility, enabling drug delivery, and so forth.

As one specific example (among many), coronary stents such as those commercially available from Boston Scientific Corp. (TAXUS and PROMUS), Johnson & Johnson (CYPHER), and others are frequently prescribed for maintaining blood vessel patency. These products are based on metallic expandable stents with biostable polymer coatings, which release antiproliferative therapeutic agents at a controlled rate and total dose for preventing restenosis of the blood vessel. One such device is schematically illustrated, for example, in FIGS. 1A and 1B. FIG. 1A is a schematic perspective view of a stent 100 which contains a number of interconnected struts 100s. FIG. 1B is a cross-section taken along line b-b of strut 100s of stent 100 of FIG. 1A, and shows a stainless steel strut substrate 110 and a therapeutic-agent-containing polymeric coating 120, which encapsulates the entire stent strut substrate 110, covering the luminal surface 110l (i.e., the inner, blood-contacting surface), the abluminal surface 110a (i.e., the outer, vessel wall-contacting surface), and side 110s surfaces thereof.

### SUMMARY OF THE INVENTION

According to one aspect, the present invention provides implantable or insertable medical devices that comprise a conductive substrate and an electrodeposited coating over the substrate that includes (a) one or more types of inorganic materials, (b) one or more types of polymeric materials and (c) optionally, one or more types of therapeutic agents.

Other aspects of the invention concern methods of making and using such devices.

The above and other aspects, as well as various embodiments and advantages of the present invention will become immediately apparent to those of ordinary skill in the art upon review of the Detailed Description and Claims to follow.

### BRIEF DESCRIPTION OF THE DRAWINGS

FIG. 1A is a schematic perspective view of a stent in accordance with the prior art. FIG. 1B is a schematic cross-sectional view taken along line b-b of FIG. 1A.

FIGS. 2A-2C are partial schematic cross-sectional views of medical devices in accordance with three embodiments of the invention.

FIGS. 3A-3C are schematic illustrations of electrochemical apparatuses for anodizing a stent and/or for forming an electrodeposited coating on a stent, in accordance with three embodiments of the present invention.

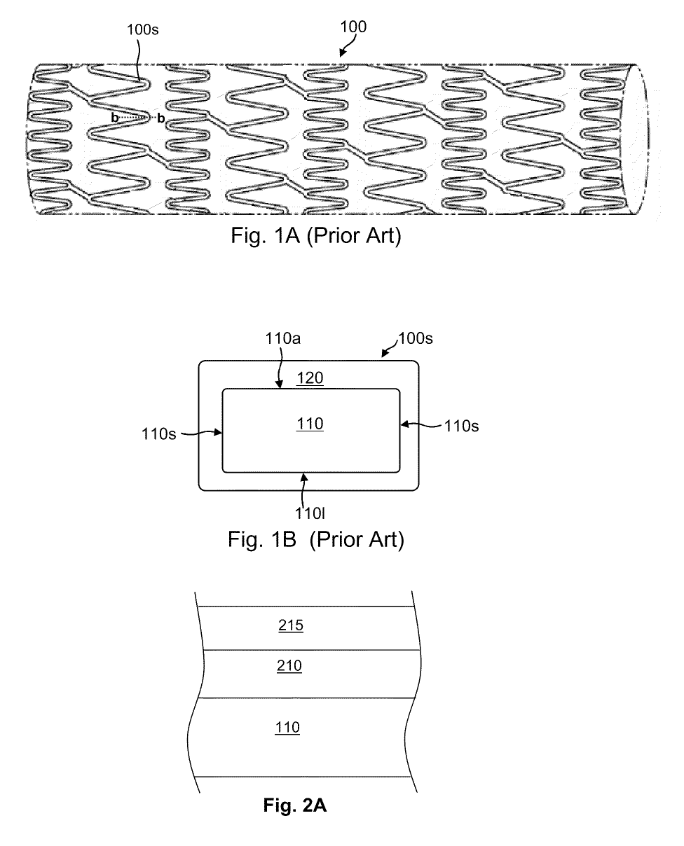

### DETAILED DESCRIPTION OF THE INVENTION

According to one aspect, the present invention provides implantable or insertable medical devices that comprise a conductive substrate and an electrodeposited coating over the substrate. The electrodeposited coating includes (a) one or more types of inorganic materials, (b) one or more types of polymeric materials and (c) optionally, one or more types of therapeutic agents.

As discussed in more detail below, the inorganic materials, polymeric materials and optional therapeutic agents can be deposited concurrently or sequentially, and they can be deposited via a number of electrodeposition mechanisms.

The electrodeposited coatings of the invention can vary widely in thickness, for example, ranging from 100 nm or less to 250 nm to 500 nm to 1 micron to 2.5 microns to 5 microns to 10 microns or more.

“Therapeutic agents”, “pharmaceuticals,” “pharmaceutically active agents”, “drugs” and other related terms may be used interchangeably herein and include genetic therapeutic agents, non-genetic therapeutic agents and cells. Therapeutic agents may be used singly or in combination. A wide variety of therapeutic agents can be employed in conjunction with the present invention, including those used for the treatment of a wide variety of diseases and conditions (i.e., the prevention of a disease or condition, the reduction or elimination of symptoms associated with a disease or condition, or the substantial or complete elimination of a disease or condition).

Examples of medical devices which can be provided with electrodeposited coatings surfaces in accordance with the invention vary widely and include implantable or insertable medical devices, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts, etc.), vascular access ports, dialysis ports, catheters (e.g. urological catheters or vascular catheters such as balloon catheters and various central venous catheters), guide wires, balloons, filters (e.g., vena cava filters and mesh filters for distil protection devices), embolization devices including cerebral aneurysm filler coils (including Guglielmi detachable coils and metal coils), embolic particles, septal defect closure devices, drug depots that are adapted for placement in an artery for treatment of the portion of the artery distal to the device, myocardial plugs, patches, pacemakers, leads including pacemaker leads, defibrillation leads and coils, neurostimulation leads such as spinal cord stimulation leads, deep brain stimulation leads, peripheral nerve stimulation leads, cochlear implant leads and retinal implant leads, ventricular assist devices including left ventricular assist hearts and pumps, total artificial hearts, shunts, valves including heart valves and vascular valves, anastomosis clips and rings, cochlear implants, tissue bulking devices, tympanostomy tubes, thoracic drainage tubes, nephrostomy tubes, and tissue engineering scaffolds for cartilage, bone, skin, nerve and other in vivo tissue regeneration, sutures, suture anchors, tissue staples and ligating clips at surgical sites, cannulae, metal wire ligatures, urethral slings, hernia “meshes”, artificial ligaments, tacks for ligament attachment and meniscal repair, joint prostheses, spinal discs and nuclei, orthopedic prosthesis such as bone grafts, bone plates, fins and fusion devices, orthopedic fixation devices such as interference screws in the ankle, knee, and hand areas, rods and pins for fracture fixation, screws and plates for craniomaxillofacial repair, dental implants, contact lenses, intraocular lenses, punctum plugs, glaucoma shunts, or other devices that are implanted or inserted into the body.

As used herein “electrodeposition” is the deposition of a material that occurs upon the application of an electrical potential between two conductive materials (or electrodes) within a liquid medium containing charged species. In various embodiments of the invention, materials are electrodeposited at the cathode (i.e., the electrode where reduction takes place). In some embodiments, the thickness of the deposited layer will vary over the surface of the device as a result of variations in current distribution during electrodeposition.

A typical apparatus for carrying out electrodeposition includes the following: an anode, a cathode and, frequently, a reference electrode, each separated by an electrolyte (e.g., an ion containing solution), as well as a potentiostat which monitors/sets the voltages/currents at the various electrodes. Electrodeposition can be carried under a variety of electrochemical conditions including the following, among others: (a) constant current, (b) constant voltage, (c) current scan/sweep, e.g., via a single or multiple scans/sweeps, (d) voltage scan/sweep, e.g., via a single or multiple scans/sweeps, (e) current square waves or other current pulse wave forms, (f) voltage square waves or other voltage pulse wave forms, and (g) a combination of different current and voltage parameters. The electrochemical techniques that use some of the listed conditions are known under different names. Common terminology for these methods include, for example, potentiostatic, potentiodynamic, potential square wave, potential square step, potential scan/hold, galvanostatic, galvanodynamic, galvanic square wave, galvanic square step and so forth.

Materials may be electrodeposited on conductive substrates by a variety of mechanisms including, for example, the following, among other mechanisms: (a) electrophoresis (e.g., migration of a positively charged species to the cathode), (b) cathodic reduction of a soluble species such that it forms an insoluble species, and (c) cathodic reactions resulting in pH gradients that cause soluble species to become insoluble.

Substrates in accordance with the present invention are at least partially conductive. For instance, a substrate may consist entirely of conductive material, may include a conductive coating layer on a non-conductive material, and so forth. Conductive materials include metallic materials and conductive polymeric materials, among others.

In many embodiments, the conductive material is a metallic material (i.e., one containing one or more metals). Examples of metallic materials include the following: (a) substantially pure metals, including gold, platinum, palladium, iridium, osmium, rhodium, titanium, zirconium, tantalum, tungsten, niobium, ruthenium, alkaline earth metals (e.g., magnesium), iron and zinc, and (b) metal alloys, including metal alloys comprising iron and chromium (e.g., stainless steels, including platinum-enriched radiopaque stainless steel), niobium alloys, titanium alloys, nickel alloys including alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), and alloys comprising nickel and chromium (e.g., inconel alloys), and metal alloys such as those described in Pub. No. US 2002/0004060 A1, entitled “Metallic implant which is degradable in vivo,” which include metal alloys whose main constituent is selected from alkali metals, alkaline earth metals, iron, and zinc, for example, metal alloys containing magnesium, iron or zinc as a main constituent and one or more additional constituents selected from the following: alkali metals such as Li, alkaline-earth metals such as Ca and Mg, transition metals such as Mn, Co, Ni, Cr, Cu, Cd, Zr, Ag, Au, Pd, Pt, Re, Fe and Zn, Group 13 metals such as Al, and Group 14 elements such as C, Si, Sn and Pb.

As noted above, electrodeposited coatings in accordance with the invention include one or more types of inorganic materials. The coatings may comprise, for example, from 5 wt % or less to 10 wt % to 25 wt % to 50 wt % to 75 wt % to 90 wt % to 95 wt % or more of one or more inorganic materials.

Inorganic materials include metallic materials and non-metallic inorganic materials. Specific examples of metallic materials for use as inorganic materials be selected, for example, from the metallic materials describe above for use as conductive substrate materials, among others.

Specific examples of non-metallic inorganic materials may be selected, for example, from materials containing one or more of the following: Periodic Table Group 14 semi-metals (e.g., C, Si, Ge); metal and semi-metal oxides, hydroxides, nitrides, carbides, oxonitrides and oxocarbides, including oxides, hydroxides, nitrides, carbides, oxonitrides and oxocarbides of Periodic Table Group 14 semi-metals; and oxides, hydroxides, nitrides, carbides, oxonitrides and oxocarbides of transition and non-transition metals such as Group 2 metals (e.g., Mg, Ca), Group 3 metals (e.g., Sc, Y), Group 4 metals (e.g., Ti, Zr, Hf), Group 5 metals (e.g., V, Nb, Ta), Group 6 metals (e.g., Cr, Mo, W), Group 7 metals (e.g., Mn, Tc, Re), Group 8 metals (e.g., Fe, Ru, Os), Group 9 metals (e.g., Co, Rh, Ir), Group 10 metals (e.g., Ni, Pd, Pt), Group 11 metals (e.g., Cu, Ag, Au), Group 12 metals (e.g., Zn, Cd, Hg), Group 13 metals (e.g., Al, Ga, In, Tl), Group 14 metals (e.g., Sn, Pb), and Group 15 metals (e.g., Bi). In certain embodiments, bioactive ceramic materials, sometimes referred to as “bioceramics,” are employed, including calcium phosphate ceramics (e.g., hydroxyapatite), calcium-phosphate glasses (sometimes referred to as glass ceramics, e.g., bioglass), and various metal oxide ceramics such as titanium oxide, iridium oxide, zirconium oxide, tantalum oxide and niobium oxide, among other materials.

In some embodiments of the invention, inorganic materials may be electrodeposited as a result of chemical or electrochemical reactions that convert soluble species into insoluble species.

For example, various metal salts (e.g., metal chlorides such as ferrous and ferric chloride salts, zirconium salts, etc.) are known to undergo cathodic deposition (i.e., deposition at a cathode) in the form of insoluble oxides and/or hydroxides. Without wishing to be bound by theory, it has been proposed that cathodic deposits of various metal oxides and/or hydroxides can be formed by hydrolyzing metal ions or complexes in basic media that is electrogenerated at the cathode. See, e.g., J. Cao et al., Materials Chemistry and Physics 96 (2006) 289-295. As one specific example among many others, I. Zhitomirsky et al., Materials Letters, 57 (2003) 1045-1050, suggest that magnetite may form from a mixture of ferrous and ferric ions in accordance with the following reaction: Fe²⁺+2Fe³⁺+8OH⁻→Fe₃O₄+4H₂O.

With respect to the electrogenerated base at the cathode, various cathodic reactions have been described which are capable of increasing solution pH (i.e., rendering it more basic) at the cathode. Commonly described examples of such reactions include, for example,

O₂+4H⁺+4e⁻→2 H₂O, where pH<7,

O₂+2 H₂O+4e⁻→4 OH⁻, where pH≧7,

2H⁺+2 e⁻→H₂, where pH<7, and

2H₂O+2e⁻→H₂+2OH⁻, where pH≧7.

(The first two reactions require the presence of oxygen, whereas the latter two do not.) Regardless of the exact mechanism, processes are known to occur at the cathode in aqueous (i.e., water containing) solutions, which can result in an increase in pH at the cathode.

In some embodiments, inorganic materials are electrodeposited as a result of electromigration of positively charged inorganic particles to the cathode.

Inorganic particles for use in the electrodeposited coatings of the invention can vary widely in size. Commonly, they are nanoparticles, meaning that they have at least one major dimension (e.g., the thickness for a nanoplates, the diameter for a nanospheres, nanocylinders and nanotubes, etc.) that is less than 1000 nm, and in certain embodiments, less than 100 nm. For example, nanoplates typically have at least one dimension (e.g., thickness) that is less than 1000 nm, other nanoparticles typically have at least two orthogonal dimensions (e.g., thickness and width for nanoribbons, diameter for nanocylinders and nanotubes, etc.) that are less than 1000 nm, while still other nanoparticles typically have three orthogonal dimensions that are less than 1000 nm (e.g., the diameter for nanospheres).

A wide variety of particles are available for use in the present invention including those formed from the above metallic and non-metallic materials. Specific examples include, for example, carbon, ceramic and metallic nanoparticles including nanoplates, nano-ribbons, nanotubes, and nanospheres, and other nanoparticles. Specific examples of nanoplates include synthetic or natural phyllosilicates including clays and micas (which may optionally be intercalated and/or exfoliated) such as montmorillonite, hectorite, hydrotalcite, vermiculite and laponite. Specific examples of nanotubes and nanofibers include single-wall, so-called “few-wall,” and multi-wall carbon nanotubes, carbon nanofibers, alumina nanofibers, titanium oxide nanofibers, tungsten oxide nanofibers, tantalum oxide nanofibers, zirconium oxide nanofibers, and silicate nanofibers such as aluminum silicate nanofibers. Further specific examples of nanoparticles (e.g., nanoparticles having three orthogonal dimensions that are less than 1000 nm) include fullerenes (e.g., “Buckey balls”), silica nanoparticles, gold nanoparticles, aluminum oxide nanoparticles, titanium oxide nanoparticles, tungsten oxide nanoparticles, tantalum oxide nanoparticles, zirconium oxide nanoparticles, iridium oxide nanoparticles, niobium oxide nanoparticles and monomeric silicates such as polyhedral oligomeric silsequioxanes (POSS), including various functionalized POSS and polymerized POSS.

In some embodiments, particles are electrodeposited via a mechanism that includes electromigration toward the cathode in the electric field that exists in the solution. In these embodiments, the particles positively charged.

Examples of charged particles include those that are inherently charged. Further examples of charged particles include those that are modified to have a charge using a suitable technique. For instance, nanoparticles may be made positively charged by applying an outer layer of a positively charged material. For example, “DNA-mediated electrostatic assembly of gold nanoparticles into linear arrays by a simple drop-coating procedure,” Murali Sastrya and Ashavani Kumar, Applied Physics Letters, Vol. 78, No. 19, 7 May 2001, 2943, describe lysine-capped colloidal gold particles. Gold nanoparticles may help to create a radio-opaque layer.

As another example, a variety of particles may be positively charged by exposure to (and adsorption of) a cationic polyelectrolyte such as poly(allyamine hydrochloride) (PAH), polyethyleneimine (PEI), poly(diallyldimethylammonium chloride) (PDDA) and chitosan, among others, including those described below. If desired, the charge on a particle can be reversed by exposing it to a solution containing a polyelectrolyte of opposite charge.

As a further example, polyelectrolytes may be covalently attached to particles (sometimes referred to as a “grafting to” approach). For instance, in R. Czerw et al., “Organization of Polymers onto Carbon Nanotubes: A Route to Nanoscale Assembly,” Nano Lett., Vol. 1, No. 8, 2001, 423-427, acyl chloride functionalized nanotubes are reacted with poly-(propionylethylenimine-co-ethylenimine) (PPEI-EI) thereby attaching the PPEI-EI to the nanotubes via amidation. As yet another specific example, N-protected amino acids have been linked to carbon nanotubes and subsequently used to attach peptides via fragment condensation or using a maleimido linker. See, e.g., S. Banerjee et al., “Covalent Surface Chemistry of Single-Walled Carbon Nanotubes,” Adv. Mater. 2007, 17, No. 1, January 6, 17-29. In this way, polycationic peptides (e.g., homopolymers and copolymers containing lysine, arginine and/or omithine) may be connected to carbon nanotubes. In another example, polyelectrolytes are polymerized from initiation sites on the surface of the particles (sometimes referred to as a “grafting from” approach).

Further examples of charged particles include those that become charged in situ in the electrodeposition environment. For example, X. Pang et al., Materials Chemistry and Physics 94 (2005) 245-251, discuss various mechanisms by which particles can become charged in situ (which will, of course, depend upon the electrodeposition environment), including (a) particle-solution exchange interactions of dissolution and ion exchange and (b) particle charging originating from charged electrolyte. As a specific example, X. Pang et al., Langmuir 20 (2004) 2921-2927, suggest that, based on a reported isoelectric point for hydrous zirconia of 6.7, colloidal zirconia particles should ordinarily be negatively charged in the basic environment near the cathode surface (and hence repelled from the cathode). These authors suggest, however, that cationic polyelectrolytes may be adsorbed to zirconia particles by electrostatic interactions or non-electrostatic interactions (e.g., by hydrogen bonding at pH values below the isoelectric point). Id.

As will be appreciated from the discussion to follow, in the case of particle charging due to polyelectrolyte attachment (e.g., due to covalent binding, adsorption, etc.), depending on the polyelectrolyte that is adsorbed, the particle may become insoluble as it migrates to the cathode due to a reduction in the solubility of the polyelectrolyte with an increase in pH. This mechanisms can be used achieve/enhance particle deposition at the cathode.

In other embodiments, neutral suspended particles of inorganic material that are present in solution at the cathode may be captured and incorporated (i.e., entrapped) during electrodeposition. For example such particles may be entrapped during electrodeposition of polymeric materials and/or optional therapeutic agents at the cathode (e.g., during the deposition of a chitosan or collagen layer as discussed below, among many other possibilities).

As previously noted, in addition to one or more types of inorganic materials, the electrodeposited coatings in accordance with the invention further include one or more types of polymeric materials. The coatings may comprise, for example, from 5 wt % or less to 10 wt % to 25 wt % to 50 wt % to 75 wt % to 90 wt % to 95 wt % or more of one or more types of polymeric materials.

As with the inorganic materials above, polymeric materials may be electrodeposited by various mechanisms including the following, among others: (a) electrophoresis (e.g., migration of polyelectrolytes, migration of charged polymer particles, etc.), (b) deposition as a result of chemical and/or electrochemical reactions that convert soluble species to insoluble species, for instance, direct reduction at the cathode (i.e., transfer of electrons to the polymeric materials) or precipitation due to a reduction in solubility at the cathode (e.g., based on pH effects), and (c) entrapment of neutrally charged polymeric materials (e.g., dissolved polymers, suspended polymer particles, etc.) during electrodeposition of inorganic materials and/or optional therapeutic agents at the cathode.

Polymeric materials for use in the coatings of the present invention can thus vary widely and may be selected, for example, from suitable members of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), poly(vinylidene fluoride-co-hexafluoropropylene) (PVDF-HFP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins and polysaccharides; as well as blends and further copolymers of the above.

Further examples of polymers include those containing poly(amino acid) sequences (e.g., linear or cyclic peptides, proteins, etc.) that pertain to cell adhesion and/or cell growth, among other effects. For example, polypeptides containing RGD sequences (e.g., GRGDS) and WQPPRARI sequences are known to direct spreading and migrational properties of endothelial cells. See V. Gauvreau et al., Bioconjug Chem., September-October 2005, 16(5), 1088-97. REDV tetrapeptide has been shown to support endothelial cell adhesion but not that of smooth muscle cells, fibroblasts, or platelets, and YIGSR pentapeptide has been shown to promote epithelial cell attachment, but not platelet adhesion. More information on REDV and YIGSR peptides can be found in U.S. Pat. No. 6,156,572 and Pub. No. US 2003/0087111. A further example of a cell-adhesive sequence is NGR tripeptide, which binds to CD13 of endothelial cells. See, e.g., L. Holle et al., “In vitro targeted killing of human endothelial cells by co-incubation of human serum and NGR peptide conjugated human albumin protein bearing alpha (1-3) galactose epitopes,” Oncol. Rep. March 2004; 11(3):613-6. Other polymers useful for cell adhesion may be selected from suitable proteins, glycoproteins, polysaccharides, proteoglycans, glycosaminoglycans and subunits and fragments of the same, for example, those set forth in Pub. No. US 2005/0187146 to Helmus et al. Specific examples of proteins include collagen, fibronectin, laminin and vitronectin, among others.

Polymers present in the coatings of the invention may be advantageous in that they act as chemical/biochemical plasticizers to offset the brittleness of certain inorganic materials, for example, ceramic materials including metal oxides.

In various embodiments, the electrodeposited polymeric materials comprise one or more polyelectrolytes. As used herein, “polyelectrolytes” are polymers having multiple (e.g., 5 to 10 to 25 to 50 to 100 to 250 to 500 to 1000 or more) charged groups (e.g., ionically dissociable groups that provide cations and anions), at least over a certain pH range. Frequently, the number of charged groups is so large that the polymers are soluble in aqueous solutions when in ionically dissociated form (also called, for example, polyions, polycations or polyanions). Polyelectrolytes may be classified as polyacids and polybases (and their salts). When dissociated, polyacids form polyanions (anionic polyelectrolytes), with protons being split off. Polybases contain groups which are capable of accepting protons, forming polycations (cationic polyelectrolytes).

Cationic polyelectrolytes include those that are positively charged at pH values of ≦5, ≦6, ≦7, ≦8, ≦9, ≦10, ≦11, ≦12, and so forth. Stronger cationic polyelectrolytes (also called strong polybases) can maintain a positive charge at greater pH values than weaker cationic polyelectrolytes. Typically, for strong cationic polyelectrolytes, the positive charge is practically independent of pH. The positive charge of weak cationic polyelectrolytes, on the other hand, is strongly dependent on the pH. For example, chitosan, discussed below, is a weak cationic polyelectrolyte. It is positively charged and water-soluble in acidic to neutral solutions, but it becomes substantially uncharged (and loses its water-solubility) at pH values of about 6.5 and above. Further examples of weak cationic polyelectrolytes include PAH and PEI, among many others. An example of a strong cationic polyelectrolyte is PDDA, among many others.

Some polyelectrolytes have both anionic and cationic groups, but nonetheless have a net negative charge, for example, because the anionic groups outnumber the cationic groups, or have a net positive charge, for example, because the cationic groups outnumber the anionic groups. In this regard, the net charge of a particular polyelectrolyte may change in sign with the pH of its surrounding environment, for example, changing (with increasing pH) from a positive net charge, to a neutral net charge (known as the isoelectric point) to a net negative charge. Polyelectrolytes containing both cationic and anionic groups may be categorized as either polycations or polyanions, depending on which groups predominate under the conditions at hand.

Thus, as defined herein, the term “polyelectrolyte” embraces a wide range of species, including polycations and their precursors (e.g., polybases, polysalts, etc.), polyanions and their precursors (e.g., polyacids, polysalts, etc.), polymers having both anionic and cationic groups (e.g., polymers having multiple acidic and basic groups such as are found in various proteins and peptides), ionomers (polyelectrolytes in which a small but significant proportion of the constitutional units carry charges), and so forth.

Specific examples of suitable polycations may be selected, for instance, from the following: polyamines, including polyamidoamines, poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines such as poly(allylamine hydrochloride), poly(diallyldialklylamines) such as poly(diallyldimethylammonium chloride), spermine, spermidine, hexadimethrene bromide(polybrene), polyimines including polyalkyleneimines such as polyethyleneimines, polypropyleneimines and ethoxylated polyethyleneimines, polycationic peptides and proteins, including histone polypeptides and homopolymer and copolymers containing lysine, arginine, omithine and combinations thereof including poly-L-lysine, poly-D-lysine, poly-L,D-lysine, poly-L-arginine, poly-D-arginine, poly-D,L-arginine, poly-L-omithine, poly-D-ornithine, and poly-L,D-omithine, gelatin, albumin, protamine and protamine sulfate, and polycationic polysaccharides such as cationic starch and chitosan, as well as copolymers, derivatives and combinations of the preceding, among various others. Certain of the above polyelectrolytes, including various bio-polyelectrolytes, are biodegradable.

As indicated above, polyelectrolytes may be electrodeposited by various mechanisms.

For example, co-electrodeposition of strong cationic polyelectrolytes and metallic oxides and/or hydroxides have been reported, including the formation of PDDA-zirconia films and PDDA-iron oxide films. See, e.g., I. Zhitomirsky et al. Materials Letters, 57 (2003) 1045-1050), X. Pang et al., Surface & Coatings Technology, 195 (2005) 138-146, and the references cited therein. Without being bound by theory, it has been suggested that deposit formation is driven by the Coulombic attraction between the positively charged PDDA and negatively charged colloidal particles (e.g., particles comprising metal oxides and/or metal hydroxides, which as indicated above are believed to be formed at the cathode in the presence of electrogenerated base). The reported thickness of the composite films was in the range of 5-10 μm. Id.

Electrodeposition of weak cationic polyelectrolytes whose charge decreases with increasing pH (specifically PAH and PEI) and metallic oxides and/or hydroxides have also been reported, including PAH-iron oxide and PEI-zirconia films. See J. Cao et al., Materials Chemistry and Physics 96 (2006) 289-295; X. Pang et al., Langmuir 20 (2004) 2921-2927. Without wishing to be bound by theory, it has been hypothesized that that the polyelectrolytes form polymer-metal ion complexes in metal salt solutions (PAH and PEI are known to form polymer-metal ion complexes) and that such polymer-metal ion complexes behave as positively charged polyelectrolytes, migrating to the cathode via electrophoresis. Id. In the higher pH environment at the cathode, free and complexed ions form insoluble metal hydroxides or oxides. Id. Moreover, the decrease in the charge of the polyelectrolyte with increasing pH reduces the electrostatic repulsion between the polyelectrolyte molecules and promotes their deposition. See also N. Nagarajan et al., Electrochimica Acta 51 (2006) 3039-3045 who posit a similar mechanism in the formation of PEI-MgOₓ films.

Regardless of the precise deposition mechanism, techniques such as the above and others can be used to created composite polyelectrolyte-inorganic coatings, which may contain one or more optional therapeutic agents.

In other embodiments of the invention, cationic polyelectrolytes are electrodeposited without concurrent electrodeposition of metal oxides/hydroxides from metal salts.

For example, chitosan has been deposited in this matter. Chitosan is a modified polysaccharide containing randomly distributed β-(1-4)-linked D-glucosamine and N-acetyl-D-glucosamine monomer units. Chitosan is produced commercially by the alkaline N-deacetylation of chitin, which is a cellulose-like polymer consisting primarily of unbranched chains of modified glucose, specifically N-acetyl-D-glucosamine. The degree of deacetylation in commercial chitosans generally ranges from 60 to 70 to 80 to 90 to 100% although essentially any degree of deacetylation is possible. Chitosan is positively charged in acidic to neutral solutions with a charge density that is dependent on the pH and the degree of deacetylation. The pka value of chitosan generally ranges from 6.1 to 7.0, depending on the degree of deacetylation. Thus, while substantially insoluble in distilled water, chitosan is generally soluble in dilute aqueous acidic solutions (e.g., pH ˜6.5 or less). Without wishing to be bound by theory, it is believed that, during electrodeposition in slightly acidic solutions, the electric field urges positively charged chitosan in the direction of the cathode. As the chitosan nears the cathode the pH increases due to the presence of electrogenerated base, causing the chitosan to lose its charge and form an insoluble deposit on the cathode surface. See, e.g., X. Pang et al., Materials Chemistry and Physics 94 (2005) 245-251.

As another example, collagen has been reported to precipitate from solution by the local pH increase that occurs at the cathode. See, e.g., Y. Fan et al., Biomaterials 26 (2005) 1623-1632. These authors further report the simultaneous deposition of calcium phosphate minerals at the cathode, and they ascribe it to supersaturation based on the local pH increase at the cathode. Id.

As noted above, in addition to one or more types of inorganic materials and one or more types of polyelectrolytes, the electrodeposited coatings in accordance with the invention may optionally further include one or more types of therapeutic agents. The coatings may comprise, for example, from 1 wt % or less to 2 wt % to 5 wt % to 10 wt % to 25 wt % or more of one or more types of therapeutic agents.

As with the inorganic materials and polymeric materials above, the optional therapeutic agents may be electrodeposited by various mechanisms including the following among others: (a) electrophoresis (e.g., migration of charged therapeutic agents, migration of charged therapeutic agents particles, etc.), (b) deposition as a result of chemical and/or electrochemical reactions that convert soluble species to insoluble species, for instance, direct reduction at the cathode (i.e., transfer of electrons) or precipitation due to a reduction in solubility at the cathode (e.g., based on pH effects), and (c) entrapment of neutral therapeutic agents (e.g., dissolved therapeutic agents, suspended therapeutic agent particles, etc.) during electrodeposition of inorganic and/or polymeric materials. The optional therapeutic agents may also be provided after be electrodeposition of a coating that includes one or more types of inorganic materials and one or more types of polymeric materials, for example, by contact with a solution that contains the therapeutic agent (e.g., by spraying, dipping, etc.)

Therapeutic agents for use in the coatings of the present invention thus vary widely.

Examples of therapeutic agents for use in connection with the present invention include: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists such as rosiglitazone, pioglitazone, netoglitazone, fenofibrate, bexaotene, metaglidasen, rivoglitazone and tesaglitazar, (bb) prostaglandin E agonists such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, and (gg) VLA-4 antagonists and VCAM-1 antagonists.

Some preferred therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and P-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein) and SOD mimics, verteporfin, rostaporfin, AGI 1067, and M40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional therapeutic agents useful for the practice of the present invention are also disclosed in U.S. Pat. No. 5,733,925 to Kunz, the entire disclosure of which is incorporated by reference.

In certain embodiments of the invention, the therapeutic is a positively charged therapeutic agent.

For example, a therapeutic agent may have an associated positive charge because it is inherently charged (e.g., because it has basic groups, which may be in salt form). A few examples of inherently charged cationic therapeutic agents include amiloride, digoxin, morphine, procainamide, and quinine, among many others.

A therapeutic agent may also have an associated positive charge because it has been chemically modified to provide it with one or more charged entities.

For instance, therapeutic agents may be conjugated to cationic species including polycationic species (e.g., weak or strong cationic polyelectrolytes). Taking paclitaxel as a specific example, various charged forms of this drug, including various cationic forms of this drug are known, including paclitaxel N-methyl pyridinium mesylate. See, e.g., U.S. Pat. No. 6,730,699; Duncan et al., Journal of Controlled Release 74 (2001)135; Duncan, Nature Reviews/Drug Discovery, Vol. 2, May 2003, 347; Jaber G. Qasem et al, AAPS Pharm Sci Tech 2003, 4(2) Article 21. U.S. Pat. No. 6,730,699 describes paclitaxel conjugated to various polyelectrolytes including poly(l-lysine), poly(d-lysine), poly(dl-lysine), poly(2-hydroxyethyl-1-glutamine) and chitosan.

T. Y. Zakharian et al., J. Am. Chem. Soc., 127 (2005) 12508-12509 describe a process for forming a fullerene-paclitaxel conjugate by covalently coupling paclitaxel-2′-succinate to a fullerene amino derivative. In the present invention, carboxylate-substituted therapeutic agents and their derivatives, including, for example, paclitaxel-2′-succinate, may be coupled to other amine-containing compounds, including, for example, polyelectrolytes such as chitosan, poly(amino acids), PEI, PAH, PDDA, etc., using a suitable linking chemistry. In many embodiments, the therapeutic agent is linked to the polyelectrolyte via a biodegradable bond. Similarly, amine-substituted therapeutic agents and their derivatives, may be coupled to carboxyl-containing compounds, including, for example, N-succinyl-chitosan.

Using the above and other strategies, paclitaxel and many other therapeutic agents may be covalently linked or otherwise associated with a variety of cationic species, including cationic polyelectrolytes, thereby forming charged drugs and prodrugs.

A therapeutic agent may also have an associated charge because it is associated with a charged particle (e.g., attached to a charged particle or forming the core of a charged particle).

Using methods such as those described above electrodeposited coatings may thus be provided that include (a) one or more types of inorganic materials, (b) one or more types of polymeric materials and (c) optionally, one or more types of therapeutic agents.

In some embodiments, the electrodeposited coatings of the invention are in the form of a single electrodeposited layer. Such embodiments may be advantageous in that interpenetrating hybrid organic-inorganic networks several microns thick may be produced during a continuous growth process.

In other embodiments, the electrodeposited coatings of the invention are formed from multiple electrodeposited layers.

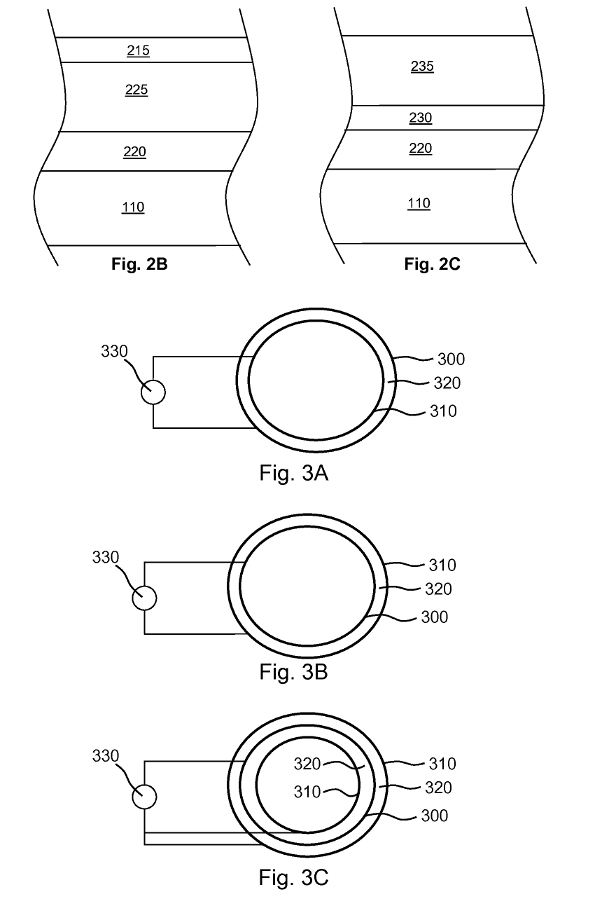

For example, referring now to FIG. 2A, a multilayer structure is shown on a substrate 110 (e.g., a metallic substrate such as stainless steel) which includes the following: a electrodeposited drug layer 210 (e.g., electrodeposited paclitaxel-chitosan conjugate, etc.) and an outer layer 215 comprising both a ceramic material (e.g., iridium oxide, titanium oxide, tantalum oxide, zirconium oxide, silicon oxide, etc.) and a polyelectrolyte (e.g., an adhesion promoting protein, etc.).

As another example, referring now to FIG. 2B, a multilayer structure is shown on a substrate 110 which includes the following: an electrodeposited ceramic layer 220, an electrodeposited ceramic and drug layer 225 (which may contain the same ceramic as layer 220 or a different ceramic), and an outer electrodeposited ceramic and polyelectrolyte layer 215.

As another example, with reference to FIG. 2C, a multilayer structure is shown on a substrate 110 which includes the following: an electrodeposited ceramic layer 220, a further electrodeposited ceramic layer 230 (containing a ceramic different from that of layer 220), and an electrodeposited layer 235 contains ceramic (which be the same ceramic as in layer 230 or a different ceramic), polymer and drug.

One additional desirable feature of the present invention is that it allows metallic substrates to be anodized (e.g., to achieve surface roughening which can improve adhesion) with essentially no further cost. This may be done, for example, by application of an appropriate anodic electrical potential while immersing the surface in a suitable electrolyte, typically an aqueous electrolytic solution. Examples of aqueous electrolytic solutions include acidic solutions (e.g., solutions of one or more of hydrochloric acid, sulfuric acid, phosphoric acid, nitric acid, among others), basic solutions (e.g., KOH, NaOH, CaOH₂, etc.), and neutral solutions (e.g., sodium nitrate, sodium chloride, potassium chloride, potassium sulfate, etc.)

FIG. 3A is a schematic illustration of an electrochemical apparatus for anodizing a tubular substrate surface (e.g., a stent surface) and/or forming an electrodeposited coating on a tubular substrate surface in accordance with an embodiment of the present invention and includes a stent 300 (end view), a cylindrical counter-electrode 310 (end view) and a suitable liquid medium 320, which is placed between the stent 300 and the counter-electrode 310. Anodization of the stent 300 or formation of an electrodeposited coating on the stent 300 is conducted via potentiostat 330. Using such an apparatus, at least the luminal surface of the stent may be anodized and/or provided with an electrodeposited coating, in accordance with the present invention.

FIG. 3B is a schematic illustration of another electrochemical apparatus for anodizing a tubular substrate surface (e.g., a stent surface) and/or forming an electrodeposited coating on a tubular substrate surface, in accordance with an embodiment of the present invention. As in FIG. 3A, a suitable liquid medium 320 and placed between the stent 300 (end view) and the counter-electrode 310 (end view) of FIG. 3B. Moreover, anodization of the stent 300 or formation of an electrodeposited coating on the stent 300 is conducted via potentiostat 330 in FIG. 3B. However, FIG. 3B is unlike FIG. 3A in that the positions of the stent 300 and the cylindrical counter-electrode 310 are reversed. Using such an apparatus, at least the abluminal surface of the stent may be anodized and/or provided with an electrodeposited coating, in accordance with the present invention.

FIG. 3C is a schematic illustration of another electrochemical apparatus for anodizing a tubular substrate surface (e.g., a stent surface) and/or forming an electrodeposited coating on a tubular substrate surface, in accordance with an embodiment of the present invention. The apparatus shown includes a stent 300 (end view), a compound counter-electrode comprising two cylindrical elements 310 (end view), and a suitable liquid medium 320, which is placed between the stent 300 and cylindrical elements 310. Anodization of the stent 300 or formation of an electrodeposited coating on the stent 300 is conducted via potentiostat 330. Using such an apparatus, at least the luminal and abluminal surface of the stent may be anodized and/or provided with an electrodeposited coating, in accordance with the present invention.

### EXAMPLE 1

A hybrid titania-paclitaxel-chitosan coating is cathodically electrodeposited on a stainless steel stent from an aqueous solution/suspension containing titanium oxide or titanium nitride nanoparticles (e.g., nanoparticles comprising titanium nitride on silica as described in R. E. Partch et al., J. Mater. Res., 8(8), 1993, 2014-2018, which may be treated with a suitable cationic polyelectrolyte or cationic surfactant to ensure a sufficient positive charge, if required), a paclitaxel-chitosan conjugate, and chitosan as a polyelectrolyte. The film is grown in a galvanostatic regime with a current density in the range of, for example, from 1-10 mA/cm². The cathodic electrodeposition process is performed at anywhere from room temperature range to about 80° C. Potentiostatic, pulsed or alternating current regimes may also be used.

Potential advantages of cathodic electrodeposition processes such as the foregoing include the following: (A) They allow the incorporation of drug and the polyelectrolyte in situ, simultaneously with the formation of ceramic oxide based coating. This process offers significant advantages of near-room temperature fabrication as well as benefits of avoiding lengthy and difficult processes in which drugs are introduced into previously formed inorganic ceramic coatings. (B) They provide the ability to control stoichiometry and to tune other physico-chemical properties of the formed coatings, such as thickness and porosity, by adjusting current density, duration, temperature, and drug, ceramic and electrolyte concentrations. (C) They are capable of producing interpenetrating hybrid networks several microns thick during a continuous growth process.

### EXAMPLE 2

The procedure of Example 1 is repeated with an everolimus-chitosan conjugate.

### EXAMPLE 3

Using a procedure analogous to like described in X. Pang et al., Langmuir 20 (2004) 2921-2927 and X. Pang et al., Surface & Coatings Technology, 195 (2005) 138-146, hybrid zirconia-polymer-paclitaxel coatings are electrodeposited on a stainless steel stent from a solution/suspension containing ZrOCl₂, a paclitaxel-poly(L-lysine) conjugate, and PAH, PEI or PDDA.

### EXAMPLE 4

The procedure of Example 3 is repeated except that, instead of paclitaxel-poly(L-lysine) conjugate, paclitaxel-PEI, paclitaxel-PAH and paclitaxel-PDDA conjugates, each having biodegradable linkages, are employed for the PEI-based deposition, PAH-based deposition, and the PDDA-based deposition, respectively.

### EXAMPLE 5

Examples 3 and 4 are repeated with analogous everolimus-polyelectrolyte conjugates.

### EXAMPLE 6

Using a procedure analogous to like described in Y. Fan et al., Biomaterials 26 (2005) 1623-1632, a hybrid zirconia-paclitaxel-collagen coating is electrodeposited on a stainless steel stent from a slightly acidic solution/suspension containing soluble type I collagen, paclitaxel-chitosan conjugate and titania nanoparticles.

### EXAMPLE 7

Example 6 is repeated except that a paclitaxel-PEI conjugate, a paclitaxel-PAH conjugate or a paclitaxel-PDDA conjugate, each having biodegradable linkages, is employed in place of the paclitaxel-chitosan conjugate.

### EXAMPLE 8

Examples 6 and 7 are repeated with analogous everolimus-polyelectrolyte conjugates.

### EXAMPLE 9

Using a procedure analogous to that described in Y. Fan et al., Biomaterials 26 (2005) 1623-1632, a hybrid hydroxyapatite-paclitaxel-collagen coating is electrodeposited on a stainless steel stent from a slightly acidic solution/suspension containing soluble type I collagen, paclitaxel-polylysine conjugate, Ca(NO₃)₂ and NH₄H₂PO₄.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device that comprises a conductive substrate and a cathodically electrodeposited coating over the substrate, said electrodeposited coating comprising an inorganic material, a polymeric material, and a therapeutic agent.

2. The implantable or insertable medical device of claim 1, wherein said conductive substrate is a metallic substrate.

3. The implantable or insertable medical device of claim 1, wherein said conductive substrate comprises a non-conductive material having a conductive coating.

4. The implantable or insertable medical device of claim 1, wherein said inorganic material is selected from metals, metal oxides, metal nitrides, calcium phosphate ceramics, and combinations thereof.

5. The implantable or insertable medical device of claim 1, wherein the electrodeposited coating comprises nanoparticles that comprise said inorganic material.

6. The implantable or insertable medical device of claim 5, wherein said nanoparticles are selected from metal oxide nanoparticles, metal nitride nanoparticles, metal carbide nanoparticles, carbon nanoparticles, and combinations thereof.

7. The implantable or insertable medical device of claim 5, wherein said nanoparticles further comprise a cationic polyelectrolyte that is covalently or non-covalently attached to the nanoparticle surface.

8. The implantable or insertable medical device of claim 1, wherein said polymeric material comprises a cationic polyelectrolyte.

9. The implantable or insertable medical device of claim 8, wherein said cationic polyelectrolyte is a biodegradable cationic polyelectrolyte.

10. The implantable or insertable medical device of claim 7, wherein the cationic polyelectrolyte is selected from chitosan, collagen, poly(allylamine hydrochloride) (PAH), polyethyleneimine (PEI) and poly(diallyldimethylammonium chloride) (PDDA).

11. The implantable or insertable medical device of claim 1, wherein said polymeric material comprises a poly(amino acid).

12. The implantable or insertable medical device of claim 11, wherein said poly(amino acid) is selected from poly(amino acids) comprising RGD polypeptide, poly(amino acids) comprising YIGSR polypeptide, and combinations thereof.

13. The implantable or insertable medical device of claim 1, wherein the therapeutic agent is an antirestenotic agent.

14. The implantable or insertable medical device of claim 1, wherein the therapeutic agent is positively charged.

15. The implantable or insertable medical device of claim 14, wherein the therapeutic agent comprises a cationic polyelectrolyte.

16. The implantable or insertable medical device of claim 14, wherein therapeutic agent comprises cationic polyelectrolyte that is conjugated to an otherwise uncharged therapeutic agent via a biodegradable linkage.

17. The implantable or insertable medical device of claim 14, wherein therapeutic agent comprises a cationic polyelectrolyte conjugated to an antirestenotic agent.

18. The implantable or insertable medical device of claim 1, wherein the coating comprises nanoparticles that comprise said therapeutic agent.

19. The implantable or insertable medical device of claim 18, wherein the nanoparticles further comprise a cationic polyelectrolyte that is covalently or non-covalently attached to the nanoparticle surface.

20. The implantable or insertable medical device of claim 1, wherein the electrodeposited coating is a single layer coating.

21. The implantable or insertable medical device of claim 1, wherein the electrodeposited coating is a multilayer coating.

22. The implantable or insertable medical device of claim 21, where said multilayer coating comprises a first cathodically electrodeposited layer comprising said therapeutic agent, and a second coating cathodically electrodeposited layer, disposed over the first cathodically electrodeposited layer, which comprises said inorganic material and said polymeric material.

23. The implantable or insertable medical device of claim 1, wherein said medical device is a stent.

24. An implantable or insertable medical device that comprises a conductive substrate and a cathodically electrodeposited coating over the substrate, said electrodeposited coating comprising an inorganic material and a protein.

25. The implantable or insertable medical device of claim 23, wherein the electrodeposited coating further comprises a therapeutic agent.
